(19) United States
(12) Patent Application Publication
(10) Pub. No.: US 2024/0408274 A1
(43) Pub. Date: Dec. 12, 2024

(54) STENTS AND METHODS OF USE

(71) Applicants: UNIVERSITY OF CONNECTICUT, Farmington, CT (US); CONNECTICUT CHILDREN'S MEDICAL CENTER, Hartford, CT (US)

(72) Inventors: Kelly Anne Burke, Sutton, MA (US); Courtney K. Rowe, West Hartford, MA (US); Chris Foster, Mystic, CT (US)

(21) Appl. No.: 18/699,213

(22) PCT Filed: Oct. 6, 2022
(86) PCT No.: PCT/US2022/045914

§ 371 Date: Apr. 5, 2024

Related U.S. Application Data
(60) Provisional application No. 63/253,114, filed on Oct. 6, 2021.

(51) Int. Cl. A61M 27/00; A61L 27/18; A61L 27/36

(52) U.S. Cl. CPC A61L 27/3604; A61L 27/18; A61M 25/0017; A61M 27/002; A61M 2025/0056; A61M 2202/0007; A61M 2205/0205; A61M 2205/0216; A61M 2205/04; A61M 2207/10; A61M 2210/1082; A61M 2210/1085

(57) ABSTRACT

Materials, methods, and techniques herein relate to stents. More specifically, the instant disclosure relates to stents, such as catheters, for slow-release drug delivery of urological therapeutic agents.

### CROSS-REFERENCE TO RELATED APPLICATION(S)

This application claims priority to U.S. Provisional Patent Application No. 63/253,114 filed on Oct. 6, 2021, the entire contents of which are incorporated herein by reference.

### TECHNICAL FIELD

Materials, methods, and techniques herein relate to stents. More specifically, the instant disclosure relates to stents, such as catheters, for slow-release drug delivery of urological therapeutic agents.

### INTRODUCTION

Urethral healing after surgery or trauma is plagued by urethral strictures and other complications (e.g., strictures, fistulas, infections, stones, renal failure). Indeed, these complications can occur in as many as 68% of patients undergoing ureteral surgery. This results in 1.5 million office visits to a medical provided each a year and/or $191 million in health care costs per year. An improved treatment for urethral healing would be more transformative for patients suffering from such complications.

### SUMMARY

In one aspect, a stent comprising a thermoplastic polyurethane (TPU) material is disclosed. A plurality of silk particles may be covalently attached to the TPU material. The silk particles may comprise a urological therapeutic agent. In some instances, the stent may be a catheter. The stent may have an elastic modulus of 1 MPa to 20 MPa. The TPU material may comprise 18% by weight (wt %) to 48 wt % poly (tetrahydrofuran). In some instances, the silk particles may be attached to the TPU material via an amide bond. The silk particles may an average diameter of 0.1 μm to 30 μm. The loading of the urological therapeutic agent may be 0.001 mg to 0.4 mg of urological therapeutic agent per 1 mg of silk particles. The urological therapeutic agent may be covalently conjugated to the silk particles. The stent may provide extended release of the urological therapeutic agent for up to 168 hours.

In another aspect, a catheter comprising a thermoplastic polyurethane (TPU) material is disclosed. A plurality of silk particles may be covalently attached to the TPU material. The silk particles may comprise a urological therapeutic agent. The catheter may have an elastic modulus of 1 MPa to 20 MPa. The loading of the urological therapeutic agent may be 0.001 mg to 0.4 mg of urological therapeutic agent per 1 mg of silk particles. The catheter may provide extended release of the urological therapeutic agent for up to 168 hours.

In another aspect, a method of manufacturing a stent is disclosed. The method may comprise enriching a thermoplastic polyurethane (TPU) material with a plurality of carboxylic acid groups. The method may further comprise covalently coupling a plurality of silk particles to the carboxylic acid groups. The silk particles may comprise a urological therapeutic agent. Enriching the TPU material with a plurality of carboxylic acid groups may comprise photooxidation of the TPU material with an oxidizing agent. Covalently coupling the silk particles to the carboxylic acid groups may comprise activating the carboxylic acid groups by reacting the groups with a carbodiimide, then reacting the silk particles with the activated carboxylic acid groups. The silk particles may comprise 0.01 wt % to 99.99 wt % amine-functionalized silk. The particles may comprise 0.01 wt % to 99.99 wt % unfunctionalized silk.

In another aspect, a method for treating a urological disease or disorder is disclosed, the method comprising implanting or inserting the stent or catheter, as described herein, into a subject in need thereof.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

FIG. 1 schematically illustrates a stent derived from thermoplastic polyurethane (TPU) with silk particles attached containing drug for drug delivery.

FIG. 2 schematically illustrates a stent placed within the urothelial lumen (urethra or ureter), where the drug diffuses against urothelium.

FIG. 3 schematically illustrates drug delivery to the urethra, ureter, or bladder based on stent location.

FIG. 4 schematically illustrates the carboxylic acid enrichment of stents, followed by carbodiimide (e.g., 1-ethyl-3-(3-dimethylaminopropyl) carbodiimide (EDC)) and N-hydroxysuccinimide (NHS) activation, for covalently attaching the silk particles to the stent.

FIG. 5 schematically illustrates an exemplary in vitro scratch assay.

FIG. 6 is a light microscope image of silk particles covalently attached to stent material.

FIGS. 7A-7B are line graphs of insulin-like growth factor 1 (IGF-1) and epidermal growth factor (EGF) wound gap coverage.

FIG. 7A is a line graph of IGF-1 wound gap coverage.

FIG. 7B is a line graph of EGF wound gap coverage.

FIG. 8 is a light microscope image of a stent material without carboxylic acid enrichment and without exposure to EDC or NHS, where the silk particles were not covalently attached and were washed away by the wash steps.

FIG. 9 is a light microscope image of a stent material without carboxylic acid enrichment but with exposure to EDC, where some silk particles were covalently attached, but most particles were washed away (only a few covalent attachment sites were present).

FIG. 10 is a light microscope image of a stent material with carboxylic acid enrichment but without exposure to EDC or NHS, so the particles were not covalently attached, and few particles are observed because they were washed away by the wash steps.

FIG. 11 is a light microscope image of a stent material with carboxylic acid enrichment and with exposure to EDC. Particles are covalently attached, and many particles are observed in this image.

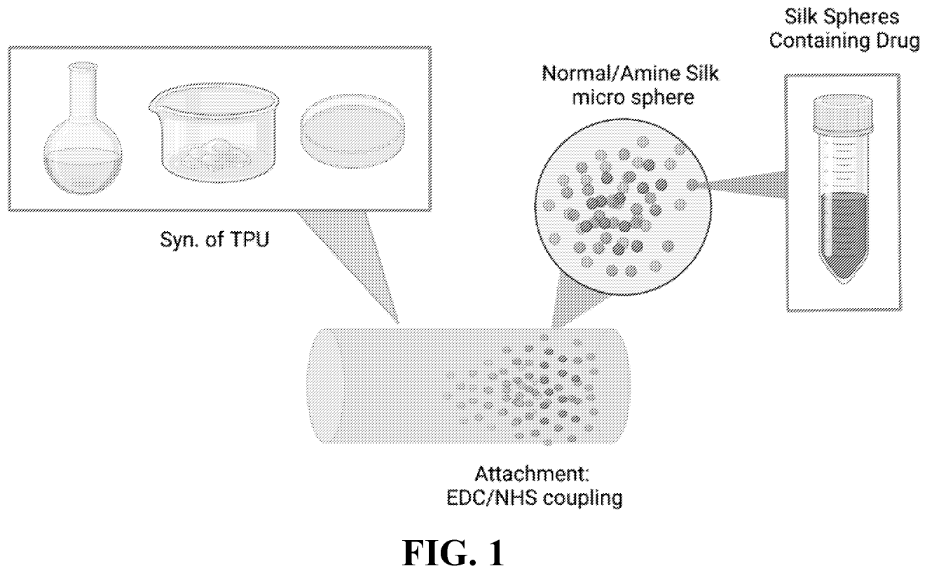

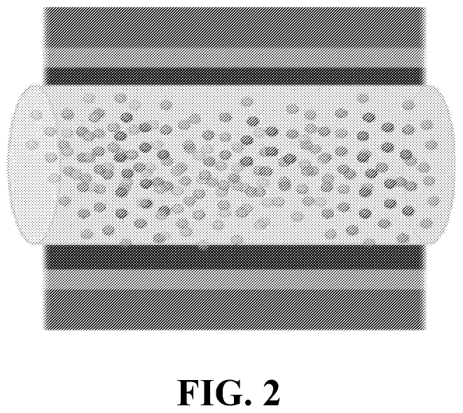

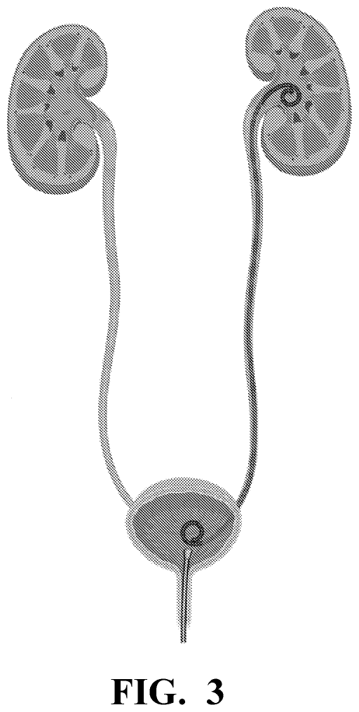

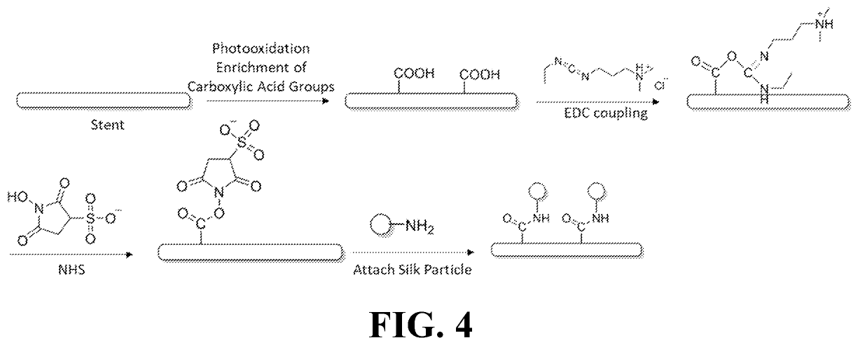

### DETAILED DESCRIPTION

Exemplary materials, methods and techniques disclosed and contemplated herein generally relate to a stent for slow release of drug to the urethra.

### I. Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” is used to indicate that exact values are not necessarily attainable. Therefore, the term “about” is used to indicate this uncertainty limit. The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5-1.4. The modifier “about” should also be considered as disclosing the range defined by the absolute values of the two endpoints. For example, the expression “from about 2 to about 4” also discloses the range “from 2 to 4.”

For the recitation of numeric ranges herein, each intervening number there between with the same degree of precision is contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated. For another example, when a pressure range is described as being between ambient pressure and another pressure, a pressure that is ambient pressure is expressly contemplated.

A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the term “silk fibroin” or “fibroin” includes silkworm fibroin and insect or spider silk protein. See, e.g., Lucas et al. 13 Adv. Protein Chem. 107 (1958). Any type of silk fibroin may be used according to aspects of the present disclosure. Silk fibroin produced by silkworms, such as Bombyx mori, is the most common and represents an earth-friendly, renewable resource. For instance, silk fibroin may be attained by extracting sericin from the cocoons of B. mori. Organic silkworm cocoons are also commercially available. There are many different silks, however, including spider silk (e.g., obtained from Nephila clavipes), transgenic silks, genetically engineered silks (recombinant silk), such as silks from bacteria, yeast, mammalian cells, transgenic animals, or transgenic plants, and variants thereof, that may be used. See, e.g., WO 97/08315 and U.S. Pat. No. 5,245,012. In some instances, silk fibroin may be derived from other sources such as spiders, other silkworms, bees, and bioengineered variants thereof. In other instances, silk fibroin may be extracted from a gland of silkworm or transgenic silkworms. See, e.g., WO 2007/098951. In various instances, silk fibroin is free, or essentially free of sericin, i.e., silk fibroin is a substantially sericin-depleted silk fibroin. The phrases “essentially free of sericin,” and “substantially sericin-depleted” may be used interchangeably to mean that no sericin is present or that sericin is present at an amount no greater than 0.0001 wt %.

As used herein an “activated carboxylic acid” is a derivative of a carboxyl group that is more susceptible to nucleophilic attack than a free carboxyl group.

As used herein a “stent” is a tubular support placed temporarily inside a canal, duct, or blood vessel to aid healing or relieve an obstruction. An exemplary stent, as described herein, is a catheter.

As used herein “Young's modulus,” also referred to as “elastic modulus,” is the resistance (MPa) of a material to elastic (recoverable) deformation under load.

### II. Compositions

Exemplary stents comprise a thermoplastic polyurethane (TPU) material and a plurality of silk particles covalently attached to the TPU material. Various aspects of exemplary TPU materials and silk particles are discussed below.

### A. Thermoplastic Polyurethane (TPU) Material

Stents described herein comprise a thermoplastic polyurethane (TPU) material. TPU was selected as the base material to achieve stents that were extensible, but flexible enough to match the properties of surrounding tissue, as well as biodegradable and biocompatible (Mi H-Y, Jing X, Napiwocki B N, et al. Biocompatible, degradable thermoplastic polyurethane based on polycaprolactone-block-polytetrahydrofuran-block-polycaprolactone copolymers for soft tissue engineering. Journal of Materials Chemistry B. 2017; 5:4137-4151). Since the TPU compositions reported in the literature have a modulus of ˜14 MPa (similar to pediatric feeding tubes), TPU materials described herein may be modified by copolymerization with an amorphous polymer to reduce crystallization to result in a programmable reduction in the material's stiffness.

In general, polyurethanes are a family of polymers that are synthesized from polyfunctional isocyanates (e.g., diisocyanates, including both aliphatic and aromatic diisocyanates) and polyols (also, referred to as macroglycols, e.g., macrodiols). Commonly employed macroglycols include polyester glycols, polyether glycols and polycarbonate glycols. Typically, aliphatic or aromatic diols are also employed as chain extenders, for example, to impart the useful physical properties described above. Examples of diol chain extenders include butane diol, pentane diol, hexane diol, heptane diol, benzene dimethanol, hydraquinone diethanol and ethylene glycol. Suitable diisocyanates include, without limitation, hexamethylene diisocyanate (HDI), isophorone diisocyanate (IPDI), trimethyl hexamethylene diisocyanate (TMHDI), dicyclohexyl methane diisocyanate (HMDI), and dimer acid diisocyanate (DDI). Suitable chain extenders include, without limitation, lower aliphatic glycols having from about 2 to about 10 carbon atoms, such as, for instance ethylene glycol, diethylene glycol, propylene glycol, dipropylene glycol, 1,4-butanediol, 1,6-hexanediol, 1,3-butanediol, 1,5-pentanediol, 1,4-cyclohexanedimethanol hydroquinone di (hydroxyethyl) ether, and neopentyglycol.

The TPU is synthesized using a block copolymer diol which contains polycaprolactone (PCL) and poly (tetrahydrofuran). To reduce the stiffness of the TPU material, additional poly (tetrahydrofuran) may be incorporated into the TPU material.

Exemplary TPU materials may comprise poly (tetrahydrofuran) at 18% by weight (wt %) to 48 wt %. In various instances, exemplary TPU materials may comprise poly (tetrahydrofuran) at 18 wt % to 45 wt %; 20 wt % to 40 wt %; or 25 wt % to 35 wt %. In various instances, exemplary TPU materials may comprise poly (tetrahydrofuran) at no greater than 18 wt %; no greater than 20 wt %; no greater than 25 wt %; no greater than 30 wt %; no greater than 35 wt %; no greater than 40 wt %; no greater than 45 wt %; or no greater than 48 wt %. In various instances, exemplary TPU materials may comprise poly (tetrahydrofuran) at no less than 18 wt %; no less than 20 wt %; no less than 25 wt %; no less than 30 wt %; no less than 35 wt %; no less than 40 wt %; no less than 45 wt %; or no less than 48 wt %.

Exemplary stents may have an elastic modulus of 1 MPa to 20 MPa. In various instances, the stent's elastic modulus may be 1 MPa to 19 MPa; 2 MPa to 18 MPa; 3 MPa to 17 MPa; 4 MPa to 16 MPa; 5 MPa to 15 MPa; 6 MPa to 14 MPa; 7 MPa to 13 MPa; 8 MPa to 12 MPa; or 9 MPa to 11 MPa. In various instances, the stent's elastic modulus may be no greater than 20 MPa; no greater than 19 MPa; no greater than 18 MPa; no greater than 17 MPa; no greater than 16 MPa; no greater than 15 MPa; no greater than 14 MPa; no greater than 13 MPa; no greater than 12 MPa; no greater than 11 MPa; no greater than 10 MPa; no greater than 9 MPa; no greater than 8 MPa; no greater than 7 MPa; no greater than 6 MPa; no greater than 5 MPa; no greater than 4 MPa; no greater than 3 MPa; no greater than 2 MPa; or no greater than 1 MPa. In various instances, the stent's elastic modulus may be no less than 1 MPa; no less than 2 MPa; no less than 3 MPa; no less than 4 MPa; no less than 5 MPa; no less than 6 MPa; no less than 7 MPa; no less than 8 MPa; no less than 9 MPa; no less than 10 MPa; no less than 11 MPa; no less than 12 MPa; no less than 13 MPa; no less than 14 MPa; no less than 15 MPa; no less than 16 MPa; no less than 17 MPa; no less than 18 MPa; no less than 19 MPa; or no less than 20 MPa.

### B. Silk Particles

Stents described herein further comprise a plurality of silk particles covalently attached to the TPU material. Exemplary silk particles may have a diameter of 0.1 μm to 30 μm. In various instances, the silk particles may have a diameter of 0.5 μm to 25 μm; 1 μm to 20 μm; 1.5 μm to 15 μm; 2 μm to 10 μm; or 2.5 μm to 5 μm. In various instances, the silk particles may have a diameter of no greater than 30 μm; no greater than 25 μm; no greater than 20 μm; no greater than 15 μm; no greater than 10 μm; no greater than 5 μm; no greater than 2.5 μm; no greater than 2 μm; no greater than 1 μm; no greater than 0.5 μm; or no greater than 0.1 μm. In various instances, the silk particles may have a diameter of no less than 0.1 μm; no less than 0.5 μm; no less than 1 μm; no less than 2 μm; no less than 2.5 μm; no less than 5 μm; no less than 10 μm; no less than 15 μm; no less than 20 μm; no less than 25 μm; or no less than 30 μm.

### C. Urological Therapeutic Agent

Silk particles attached to the TPU materials described herein comprise a urological therapeutic agent.

Examples of urological therapeutic agent(s) include, without limitation, a therapeutic agent, or a biological material, such as cells (including stem cells such as induced pluripotent stem cells), proteins, peptides, nucleic acids (e.g., DNA, RNA, siRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, viruses, antivirals, toxins, therapeutic agents and prodrugs, small molecules and any combinations thereof. See, e.g., WO 2009/140588; U.S. Patent Application Ser. No. 61/224,618). The urological therapeutic agent may also be a combination of any of the above-mentioned agents. Encapsulating either a therapeutic agent or biological material, or the combination of them, is desirous because the encapsulated composition may be used for numerous biomedical purposes.

In various instances, the urological therapeutic agent may also be an organism such as a fungus, plant, animal, bacterium, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins, and radiopharmaceuticals.

Exemplary cells suitable for use herein may include, but are not limited to, progenitor cells or stem cells (including, e.g., induced pluripotent stem cells), smooth muscle cells, skeletal muscle cells, cardiac muscle cells, epithelial cells, endothelial cells, urothelial cells, fibroblasts, myoblasts, ocular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents may also be the combinations of any of the cells listed above. See also WO 2008/106485; WO 2010/040129; WO 2007/103442.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins, and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants, and analogs of either RNA or DNA made from nucleotide analogs, and single (sense or antisense) and double-stranded polynucleotides. The term “nucleic acid” also encompasses modified RNA (modRNA). The term “nucleic acid” also encompasses siRNA, shRNA, or any combinations thereof.

The term “modified RNA” means that at least a portion of the RNA has been modified, e.g., in its ribose unit, in its nitrogenous base, in its internucleoside linkage group, or any combinations thereof. Accordingly, in various instances, a “modified RNA” may contain a sugar moiety which differs from ribose, such as a ribose monomer where the 2′-OH group has been modified. Alternatively, or in addition to being modified at its ribose unit, a “modified RNA” may contain a nitrogenous base which differs from A, C, G and U (a “non-RNA nucleobase”), such as T or MeC. In various instances, a “modified RNA” may contain an internucleoside linkage group which is different from phosphate (—O—P(O)₂—O—), such as —O—P(O,S)—O—. In various instances, a modified RNA may encompass locked nucleic acid (LNA).

The term “short interfering RNA” (siRNA), also referred to herein as “small interfering RNA” is defined as an agent which functions to inhibit expression of a target gene, e.g., by RNAi. An siRNA may be chemically synthesized, it may be produced by in vitro transcription, or it may be produced within a host cell. siRNA molecules may also be generated by cleavage of double stranded RNA, where one strand is identical to the message to be inactivated. The term “siRNA” refers to small inhibitory RNA duplexes that induce the RNA interference (RNAi) pathway. These molecules may vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that may form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi may result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term may include naturally occurring enzymes and bioengineered enzymes or mixtures thereof. Examples of enzyme families include, but are not limited to, peroxidase, lipase, amylose, organophosphate dehydrogenase, ligases, restriction endonucleases, ribonucleases, DNA polymerases, glucose oxidase, laccase, kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded, or double-stranded nucleotide sequence capable of specifically recognizing a selected non-oligonucleotide molecule or group of molecules. In various instances, the aptamer recognizes the non-oligonucleotide molecule or group of molecules by a mechanism other than Watson-Crick base pairing or triplex formation. Aptamers may include, without limitation, defined sequence segments and sequences comprising nucleotides, ribonucleotides, deoxyribonucleotides, nucleotide analogs, modified nucleotides and nucleotides comprising backbone modifications, branchpoints and non-nucleotide residues, groups, or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments may be produced by recombinant DNA techniques or by enzymatic or chemical cleavage of intact antibodies. “Antigen-binding fragments” include, inter alia, Fab, Fab′, F (ab′) 2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F (ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or may be raised against these cell-surface markers by methods known to those skilled in the art.

Exemplary antibodies that may be used, include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab. The active agents may also be the combinations of any of the antibodies listed above.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-17 (1987)). In some instances, a complementarity determining region may include amino acids from both a CDR region defined according to Kabat and a hypervariable loop.

The expression “linear antibodies” refers to the antibodies described in Zapata et al., Protein Eng., 8 (10): 1057-1062 (1995). Briefly, these antibodies comprise a pair of tandem Fd segments (VH-CH1-VH-CH1) which, together with complementary light chain polypeptides, form a pair of antigen binding regions. Linear antibodies may be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, PO: 6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” or “antimicrobial compound” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics may include, but are not limited to, actinomycin; aminoglycosides (e.g., neomycin, gentamicin, tobramycin); B-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents may also be the combinations of any of the agents listed above. See also PCT/US2010/026190.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” may also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen may have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug” or a “vaccine.” This term includes externally and internally administered topical, localized, and systemic human and animal pharmaceuticals, treatments, remedies, nutraceuticals, cosmeceuticals, biologicals, devices, diagnostics, and contraceptives, including preparations useful in clinical and veterinary screening, prevention, prophylaxis, healing, wellness, detection, imaging, diagnosis, therapy, surgery, monitoring, cosmetics, prosthetics, forensics, and the like. This term may also be used in reference to agricultural, workplace, military, industrial and environmental therapeutics, or remedies comprising selected molecules or selected nucleic acid sequences capable of recognizing cellular receptors, membrane receptors, hormone receptors, therapeutic receptors, microbes, viruses, or selected targets comprising or capable of contacting plants, animals and/or humans. This term may also specifically include nucleic acids and compounds comprising nucleic acids that produce a bioactive effect, for example deoxyribonucleic acid (DNA), ribonucleic acid (RNA), modified DNA or RNA, or mixtures or combinations thereof, including, for example, DNA nanoplexes.

The term “therapeutic agent” also includes an agent that is capable of providing a local or systemic biological, physiological, or therapeutic effect in the biological system to which it is applied. For example, the therapeutic agent may act to control infection or inflammation, enhance cell growth and tissue regeneration, control tumor growth, act as an analgesic, promote anti-cell attachment, and enhance bone growth, among other functions. Other suitable therapeutic agents may include anti-viral agents, hormones, antibodies, or therapeutic proteins. Other therapeutic agents include prodrugs, which are agents that are not biologically active when administered but, upon administration to a subject are converted to biologically active agents through metabolism or an alternative mechanism. Additionally, a silk-based composition may contain combinations of two or more therapeutic agents.

In various instances, different types of therapeutic agents that may be encapsulated or dispersed in a silk fibroin-based material may include, but not limited to, proteins, peptides, antigens, immunogens, vaccines, antibodies, or portions thereof, antibody-like molecules, enzymes, nucleic acids, modified RNA, siRNA, shRNA, aptamers, small molecules, antibiotics, and any combinations thereof.

Exemplary therapeutic agents include, but are not limited to, those found in Harrison's Principles of Internal Medicine, 13th Edition, Eds. T. R. Harrison et al. McGraw-Hill N.Y., NY; Physicians Desk Reference, 50th Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Exemplary therapeutic agents, include, but are not limited to, a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally alpha-agonist, active an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent may be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; anti-inflammatory agents, including antiasthmatic anti-inflammatory agents, antiarthritis anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, bupivacaine, ropivacaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; or nucleic acids such as DNA sequences encoding for biological proteins, and antisense oligonucleotides.

Exemplary anti-cancer agents include, but are not limited to, alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin receptor antagonists, retinoic acid receptor agonists, immuno-modulators, hormonal and antihormonal agents, photodynamic agents, and tyrosine kinase inhibitors.

Exemplary antibiotics include, but are not limited to, aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Exemplary enzyme inhibitors include, but are not limited to, edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N′-monomethyl-Larginine acetate, carbidopa, 3-hydroxybenzylhydrazine, hydralazine, clorgyline, deprenyl, hydroxylamine, iproniazid phosphate, 6-Me0-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4, 5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Exemplary antihistamines include, but are not limited to, pyrilamine, chlorpheniramine, and tetrahydrazoline.

Exemplary anti-inflammatory agents include, but are not limited to, corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Exemplary muscle relaxants include, but are not limited to, mephenesin, methocarbomal, cyclobenzaprine hydrochloride, trihexylphenidyl hydrochloride, levodopa/carbidopa, and biperiden.

Exemplar anti-spasmodics include, but are not limited to, atropine, scopolamine, oxyphenonium, and papaverine.

Exemplary analgesics include, but are not limited to, aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocaine, tetracaine, bupivacaine, ropivacaine, and dibucaine.

Exemplary ophthalmic agents include, but are not limited to, sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related, long-chain hydroxy fatty acids that have a variety of biological effects.

Anti-depressants are substances capable of preventing or relieving depression. Exemplary anti-depressants include, but are not limited to, imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. Exemplary trophic factors include, but are not limited to, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Exemplary hormones include, but are not limited to, estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g., testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

The urological therapeutic agent may be loaded onto the silk particles before or after said particles are attached to the TPU material.

In various instances, the loading of the urological therapeutic agent is 0.001 mg to 0.40 mg of urological therapeutic agent per 1 mg of silk particles. In various instances, for each 1 mg of silk particles, the loading of the urological therapeutic agent is 0.005 mg to 0.35 mg; 0.01 mg to 0.30 mg; 0.05 mg to 0.25 mg; or 0.10 mg to 0.20 mg. In various instances, for each 1 mg of silk particles, the loading of the urological therapeutic agent is no greater than 0.40 mg; no greater than 0.35 mg; no greater than 0.30 mg; no greater than 0.25 mg; no greater than 0.20 mg; no greater than 0.15 mg; no greater than 0.10 mg; no greater than 0.05 mg; no greater than 0.01 mg; no greater than 0.005 mg; or no greater than 0.001 mg. In various instances, for each 1 mg of silk particles, the loading of the urological therapeutic agent is no less than 0.001 mg; no less than 0.005 mg; no less than 0.01 mg; no less than 0.05 mg; no less than 0.1 mg; no less than 0.15 mg; no less than 0.20 mg; no less than 0.25 mg; no less than 0.30 mg; no less than 0.35; or no less than 0.40 mg.

Exemplary stents may provide extended release of the urological therapeutic agent for up to 168 hours. In various instances, the stent provides extended release of the urological therapeutic agent over a period of 5 hours to 165 hours; 10 hours to 160 hours; 15 hours to 155 hours; 20 hours to 150 hours; 25 hours to 145 hours; 30 hours to 140 hours; 35 hours to 135 hours; 40 hours to 130 hours; 45 hours to 125 hours; 50 hours to 120 hours; 55 hours to 115 hours; 60 hours to 110 hours; 65 hours to 105 hours; 70 hours to 100 hours; 75 hours to 95 hours; or 80 hours to 90 hours. In various instances, the stent provides extended release of the urological therapeutic agent over a period of no greater than 168 hours; no greater than 165 hours; no greater than 160 hours; no greater than 155 hours; no greater than 150 hours; no greater than 145 hours; no greater than 140 hours; no greater than 135 hours; no greater than 130 hours; no greater than 125 hours; no greater than 120 hours; no greater than 115 hours; no greater than 110 hours; no greater than 105 hours; no greater than 100 hours; no greater than 95 hours; no greater than 90 hours; no greater than 85 hours; no greater than 80 hours; no greater than 75 hours; no greater than 70 hours; no greater than 65 hours; no greater than 60 hours; no greater than 55 hours; no greater than 50 hours; no greater than 45 hours; no greater than 40 hours; no greater than 35 hours; no greater than 30 hours; no greater than 25 hours; no greater than 20 hours; no greater than 15 hours; no greater than 10 hours; or no greater than 5 hours. In various instances, the stent provides extended release of the urological therapeutic agent over a period of no less than 5 hours; no less than 10 hours; no less than 15 hours; no less than 20 hours; no less than 25 hours; no less than 30 hours; no less than 35 hours; no less than 40 hours; no less than 45 hours; no less than 50 hours; no less than 55 hours; no less than 60 hours; no less than 65 hours; no less than 70 hours; no less than 75 hours; no less than 80 hours; no less than 85 hours; no less than 90 hours; no less than 95 hours; no less than 100 hours; no less than 105 hours; no less than 110 hours; no less than 115 hours; no less than 120 hours; no less than 125 hours; no less than 130 hours; no less than 135 hours; no less than 140 hours; no less than 145 hours; no less than 150 hours; no less than 155 hours; no less than 160 hours; no less than 165 hours; or no less than 168 hours.

### III. Methods

Exemplary methods can be used to manufacture stents. Various aspects of methods of preparing stents are described below.

Exemplary methods of manufacturing stents according to the present disclosure may comprise enriching a thermoplastic polyurethane (TPU) material with a plurality of carboxylic acid groups. Enriching the TPU material with a plurality of carboxylic acid groups may comprise photooxidation of the TPU material with an oxidizing agent. In various instances, the oxidizing agent may be ammonium persulfate (APS)

Exemplary methods of manufacturing stents according to the present disclosure may further comprise covalently coupling a plurality of silk particles to the carboxylic acid groups, wherein the silk particles comprise a urological therapeutic agent.

Exemplary silk particles may comprise up to 100% amine functionalized silk. In some instances, the silk particles may comprise amine functionalized silk at 0.01% by weight (wt %) to 99.99 wt %. In various instances, the silk particles may comprise amine functionalized silk at 0.1 wt % to 99.9 wt %; 1 wt % to 99 wt %; 5 wt % to 95 wt %; 10 wt % to 90 wt %; 15 wt % to 85 wt %; 20 wt % to 80 wt %; 25 wt % to 75 wt %; 30 wt % to 70 wt %; 35 wt % to 65 wt %; 40 wt % to 60 wt %; or 55 wt % to 65 wt %. In various instances, the silk particles may comprise amine functionalized silk at no greater than 99.99 wt %; no greater than 99.9 wt % no greater than 99 wt % no greater than 95 wt %; no greater than 85 wt %; no greater than 75 wt %; no greater than 65 wt %; no greater than 55 wt %; no greater than 45 wt %; no greater than 35 wt %; no greater than 25 wt %; no greater than 15 wt %; no greater than 5 wt %; no greater than 1 wt %; no greater than 0.1 wt %; or no greater than 0.01 wt %. In various instances, the silk particles may comprise amine functionalized silk at no less than 0.01 wt %; no less than 0.1 wt %; no less than 1 wt %; no less than 5 wt %; no less than 15 wt %; no less than 25 wt %; no less than 35 wt %; no less than 45 wt %; no less than 55 wt %; no less than 65 wt %; no less than 75 wt %; no less than 85 wt %; no less than 95 wt %; no less than 99 wt %; no less than 99.9 wt %; or no less than 99.99 wt %.

Exemplary silk particles may comprise up to 100% unfunctionalized silk. In some instances, the silk particles may comprise unfunctionalized silk at 0.01 wt % to 99.99 wt %. In various instances, the silk particles may comprise unfunctionalized silk at 0.1 wt % to 99.9 wt %; 1 wt % to 99 wt %; 5 wt % to 95 wt %; 10 wt % to 90 wt %; 15 wt % to 85 wt %; 20 wt % to 80 wt %; 25 wt % to 75 wt %; 30 wt % to 70 wt %; 35 wt % to 65 wt %; 40 wt % to 60 wt %; or 45 wt % to 55 wt %. In various instances, the silk particles may comprise unfunctionalized silk at no greater than 95 wt %; no greater than 85 wt %; no greater than 75 wt %; no greater than 65 wt %; no greater than 55 wt %; no greater than 45 wt %; no greater than 35 wt %; no greater than 25 wt %; no greater than 15 wt %; or no greater than 5 wt %. In various instances, the silk particles may comprise unfunctionalized silk at no less than 0.01 wt %; no less than 0.1 wt %; no less than 1 wt %; no less than 5 wt %; no less than 15 wt %; no less than 25 wt %; no less than 35 wt %; no less than 45 wt %; no less than 55 wt %; no less than 65 wt %; no less than 75 wt %; no less than 85 wt %; no less than 95 wt %; no less than 99 wt %; no less than 99.9 wt %; or no less than 99.99 wt %.

Covalent coupling of the silk particles to the carboxylic acid groups may comprise activating the carboxylic acid groups by reacting the groups with a carbodiimide, followed by reacting the silk particles with the activated carboxylic acid groups. Suitable carbodiimides include, without limitation, 1-ethyl-3-(3-dimethylaminopropyl) carbodiimide (EDC)), diisopropylcarbodiimide (DIC), and dicyclohexylcarbodiimide (DCC).

### IV. Applications

Exemplary methods for treating a urological disease or disorder may comprise implanting or inserting stents of the present disclosure into a subject in need thereof.

### V. Experimental Examples

Without limiting the scope of the instant disclosure, various experimental examples of embodiments discussed above were prepared and the results are discussed below.

### Example 1: Preparation of Silk Fibroin Solution

To prepare silk fibroin particles, a silk fibroin solution was prepared according to Rockwood, D., Preda, R., Yücel, T. et al. Materials fabrication from Bombyx mori silk fibroin. Nat Protocols. 2011:6, 1612-1631.

### Example 2: Generation of Silk Fibroin Functionalized with Amine Groups

To generate silk fibroin particles functionalized with amine groups 4-(2-aminoethyl) aniline was dissolved in acetonitrile at a concentration of 0.2 M. p-Toluenesulfonic acid was dissolved in deionized water at a concentration of 1.6 M. Sodium nitrite was dissolved in deionized water at a concentration of 0.8 M. All reagents were cooled for at least 45 minutes by placing on an ice bath. Next, 1.5 mL of the 4-(2-aminoethyl) aniline solution was combined with 0.625 mL of the p-toluenesulfonic acid solution and 0.625 mL of the sodium nitrite solution and mixed by brief vortexing. The mixture was returned to the ice bath to react for 10-30 minutes. This solution is referred to as the diazonium salt solution.

To react the diazonium salt solution with silk fibroin, a silk fibroin solution in borate buffer was used. The borate buffer was composed of 100 mM sodium borate and 150 mM sodium chloride at pH 9. The silk concentration in the buffer was 5 grams of silk per 100 mL solution. The silk solution (2 mL) was combined with varying volumes (0.1 mL to 0.5 mL) of the diazonium salt solution, and the mixture was kept on ice to react for up to 30 minutes. The mixture was purified by passing through Sephadex size exclusion columns using deionized water as the eluent. All concentrations of silk were 5 grams of silk per 100 mL of solution.

Polyvinyl alcohol (PVA) was dissolved in water at a concentration of 5 grams per 100 mL. The selected silk fibroin solution was used at a concentration of 5 g silk per 100 ml of water. The PVA solution (4 mL) was mixed with 1 mL of the silk solution. The entire solution was cast in a plastic casting dish (about 60 mm in diameter) and allowed to dry at room temperature overnight. The dried film was then placed in a beaker with 30 mL deionized water and allowed to dissolve, which resulted in the formation of particles. The solution was transferred into a centrifuge tube, where it was centrifuged (9000 RPM, 20 min, 4° C.) and the supernatant was removed. Fresh water (30 mL) was then added to the particles, which were resuspended by pipetting. The suspension was then centrifuged again, and the supernatant removed. This washing process was repeated for a total of four times. The result is an aqueous solution of silk particles. Sonication of the solution at varying intensity can be used to change the particle size and distribution. The particles can be placed in different buffers by adding an additional centrifugation step and replacing the deionized water with a suitable buffer, such as phosphate buffered saline.

### Example 3: Synthesis of Stent Materials

A prepolymer was prepared by reacting hexamethylene diisocyanate (HDI) for 3 hours with a block copolymer diol (polycaprolactone-block-polytetrahydrofuran-block-polycaprolactone) using dibutyltin dilaurate. Next, butanediol was added to the reaction and allowed to react for 3 hours to generate the final polyurethane polymer. The reaction was precipitated in water and the polymer was isolated by filtration. The polymer was then soaked in isopropanol for 2 days before drying under vacuum at 50° C. for 5 days. The stents can be prepared from the polymer by thermal molding or by solvent casting from a suitable solvent, such as chloroform.

### Example 4: Tuning of Stent Materials

The mechanical properties of the polyurethane materials can be changed by polymerizing adding additional polytetrahydrofuran into the copolymer reaction.

To reduce the stiffness of the stent larger amounts of poly (tetrahydrofuran) were incorporated into the TPU polymer. The table below shows the stiffness and the strain at fracture for a series of polymer samples. The initial condition contains no additional poly (tetrahydrofuran), but the “softened” polymers contain varying weight percents (3-12 wt %) of poly (tetrahydrofuran) (Table 1). The materials were tested using rectangular film specimens, to allow comparison of the material without impact from the geometry of the stent.

TABLE 1
TPU material and Corresponding Young's
Modulus (i.e., Elastic Modulus)

| | Young's | Strain at |
|---|---|---|
| Material | Modulus (MPa) | Fracture (%) |
| Initial Polymer | 14.0 | 250 |
| 3 wt % poly(tetrahydrofuran) | 13.29 | 245 |
| 5 wt % poly(tetrahydrofuran) | 12.74 | 241 |
| 8 wt % poly(tetrahydrofuran) | 10.83 | 248 |
| 12 wt % poly(tetrahydrofuran) | 4.87 | 157 |

### Example 5: Enrichment of Carboxylic Acid Attachment Sites on Stent

A 15 wt % ammonium persulfate solution (APS) was prepared in deionized water. The stent was soaked in this solution and placed under 254 nm ultraviolet light for 15 minutes to generate hydroxyl and carboxylic acid groups on the surface of the stent. Holding the stent under the UV light for longer may result in more or less enrichment (this also depends on the power of the lamp). Residual solution was removed from the stent, and the stent was washed with deionized water for 30 minutes three times.

### Example 6: Loading Dye and/or Drug During Silk Particle Synthesis

First, a silk solution was prepared. Unfunctionalized silk fibroin, amine-enriched silk fibroin, or a mixture thereof may be present the silk solution. All concentrations of silk were 5 grams of silk per 100 mL of solution.

Polyvinyl alcohol was dissolved in water at a concentration of 5 grams per 100 mL. The silk fibroin solution was used at a concentration of 5 g per 100 ml of water. 4 mL of the PVA solution was mixed with 1 mL of the silk solution, and 1 mL of a 1 mg/mL dye or drug dissolved in water was added to the solution as well. The entire solution was cast in a plastic casting dish (about 60 mm in diameter) and allowed to dry at room temperature overnight. The dried film was then placed in a beaker with 30 mL deionized water and allowed to dissolve, which resulted in the formation of particles. The solution was transferred into a centrifuge tube, where it was centrifuged (9000 RPM, 20 min, 4° C.) and the supernatant was removed. Fresh water (30 mL) was then added to the particles, which were resuspended by pipetting. The suspension was then centrifuged again, and the supernatant was removed. This washing process was repeated for a total of four times. The result is an aqueous solution of silk particles containing drug. Sonication of the solution at varying intensity can be used to change the particle size and distribution. The particles can be placed in different buffers by adding an additional centrifugation step and replacing the deionized water with a suitable buffer, such as phosphate buffered saline.

Optionally, the silk particles may be further washed with a silk solution. The optional silk solution wash can provide an additional barrier to dye or drug diffusion. Specifically, the silk particles can be washed with varying amounts, e.g., 0.5-5% (weight/volume), of silk fibroin in water. As described above, the silk fibroin may comprise amine functionalized silk fibroin, unfunctionalized silk fibroin, or a combination thereof. The particles can then be centrifuged, and the supernatant discarded. The particles can then be resuspended in a methanol-water solution that range from 50-95 vol % methanol. Other alcohols may be substituted for methanol (such as ethanol, propanol, or butanol) to induce beta sheet formation in the silk. After this process is completed, the particles are centrifuged, and the supernatant discarded. The particles are then washed four times with water or buffer solution to remove residual alcohol.

### Example 7: Loading Dye and/or Drug after Silk Particle Synthesis

First, a silk solution was prepared. Unfunctionalized silk fibroin, amine-enriched silk fibroin, or a mixture thereof may be present the silk solution. All concentrations of silk were 5 grams of silk per 100 mL of solution.

Polyvinyl alcohol (PVA) was dissolved in water at a concentration of 5 grams per 100 mL. The selected silk fibroin solution was used at a concentration of 5 g per 100 ml of water. 4 mL of the PVA solution was mixed with 1 mL of the silk solution. The entire solution was cast in a plastic casting dish (about 60 mm in diameter) and allowed to dry at room temperature overnight. The dried film was then placed in a beaker with 30 mL deionized water and allowed to dissolve, which resulted in the formation of particles. The solution was transferred into a centrifuge tube, where it was centrifuged (9000 RPM, 20 min, 4° C.) and the supernatant was removed. Fresh water (30 mL) was then added to the particles, which were resuspended by pipetting. The suspension was then centrifuged again, and supernatant removed. This washing process was repeated for a total of four times, resulting in an aqueous solution of silk particles. Sonication of the silk particle solution at varying intensity can be used to change the particle size and distribution. The particles can be placed in different buffers by adding an additional centrifugation step and replacing the deionized water with a suitable buffer, such as phosphate buffered saline.

To load the particles with drug, the particles are centrifuged, and the supernatant is removed. The dye or drug is dissolved in deionized water or suitable aqueous buffer (such as phosphate buffered saline) at a desired concentration (e.g., 0.1-1 mg/mL). The particles are resuspended in the dye or drug solution, which are incubated overnight at 4° C. (if drug) or room temperature (if dye). Fresh water (30 mL) was then added to the particles, which were resuspended by pipetting. The suspension was then centrifuged, and the supernatant was removed. This washing process was repeated for a total of four times. The result is an aqueous solution of silk particles loaded with drug or dye. The particles can be placed in different buffers by adding an additional centrifugation step and replacing the deionized water with a suitable buffer, such as phosphate buffered saline.

Optionally, the silk particles may be further washed with a silk solution wash. The optional silk solution wash may provide an additional barrier to dye or drug diffusion. Specifically, for the optional wash, the silk particles are washed with varying amounts, such as 0.5-5% (weight/volume), of silk fibroin in water. As described above, the silk fibroin may comprise amine functionalized silk fibroin, unfunctionalized silk fibroin, or a combination thereof. The particles can then be centrifuged, and the supernatant discarded. The particles can then be resuspended in a methanol-water solution that range from 50-95% (volume percent) methanol. Other alcohol solvents may be substituted for methanol (such as ethanol, propanol, or butanol) to induce beta sheet formation in the silk. After this process is completed, the particles are centrifuged, and the supernatant discarded. The particles are then washed four times with water or buffer solution to remove residual alcohol.

### Example 8: Attachment of Silk Particles to Stent

The stent was soaked in 0.1 M MES (2-ethanesulfonic acid) buffer at a pH of 4.5-5.0 for 30 minutes. EDC (1-ethyl-3-(3-dimethylaminopropyl) carbodiimide) was added to the buffer at a concentration of 2 mg/mL and NHS (N-hydroxysuccinimide) was added to the buffer at a concentration of 3 mg/mL. The stent was incubated in this EDC/NHS solution for 15 minutes before transferring to a solution of silk fibroin particles suspended in phosphate buffered saline at a pH of 7. The stent was allowed to react with the silk particles for 2 hours to covalently attach the particles to the stent. Finally, the stent with covalently attached particles was washed three times with phosphate buffered saline.

### Example 9: In Vitro Model of Growth Factor (GF) Delivery

To deliver a growth factor (GF) from the new material, silk microparticles were synthesized using an established protocol as described by Wang et al. (J. Control Release 2007; 117: pp. 360-370) and Kaplan et al. (US 2010/0028451). These microparticles have been demonstrated utility for the slow-release fibroblast growth factor (FGF; Qu et al. Materials (Basel) 2018; 11). Prior studies have also used poly lactic-co-glycolic acid (PLGA) nanoparticles for slow release. However, PGLA creates an acidic environment during degradation and so was rejected due to concern for impact on urethral healing (Fu K, Pack D W, Klibanov A M, et al. Visual evidence of acidic environment within degrading poly (lactic-co-glycolic acid) (PLGA) microparticles. Pharmaceutical research. 2000; 17: pp. 100-106). Instead, silk microparticles of different sizes slowly degrade, releasing the GF into the urethral lumen for diffusion through the urethral layers. Drugs placed within the urethral lumen with intact urothelium are able to diffuse through the submucosa to reach the muscularis within 24 hours of urethral delivery. Therefore, when the urethral epithelium is already disrupted from surgery or trauma, the degrading silk microparticles should easily provide targeted, slow delivery of the GF.

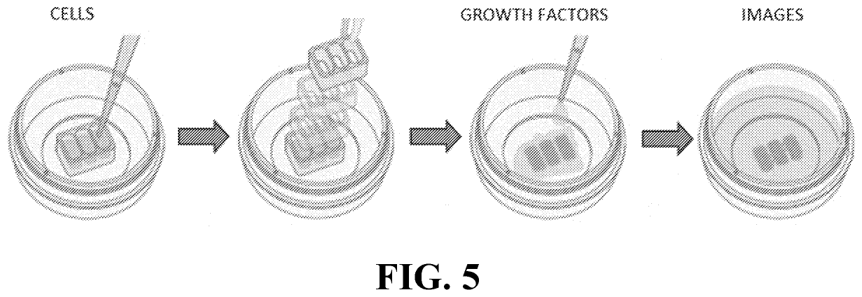

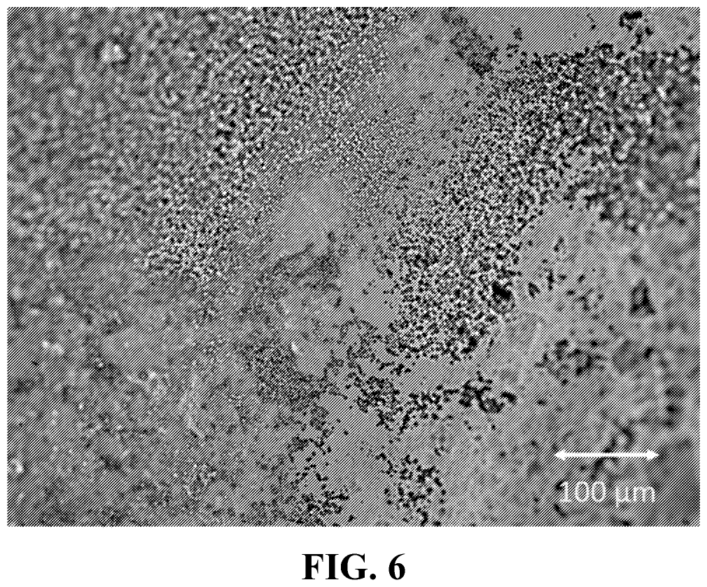

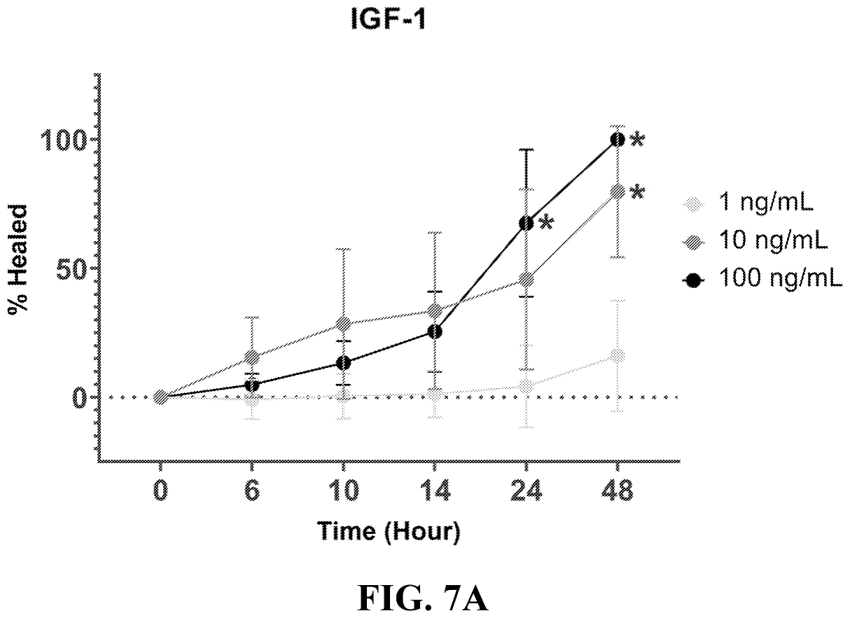

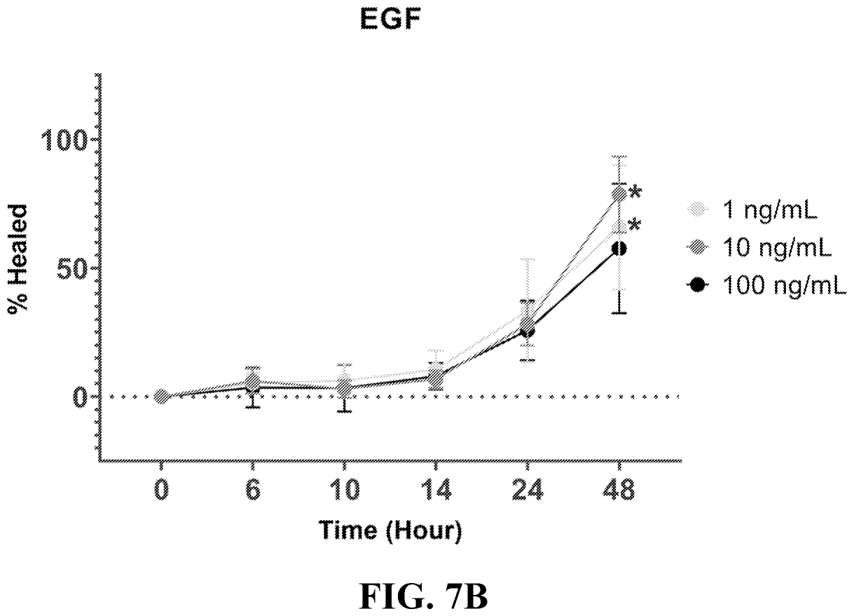

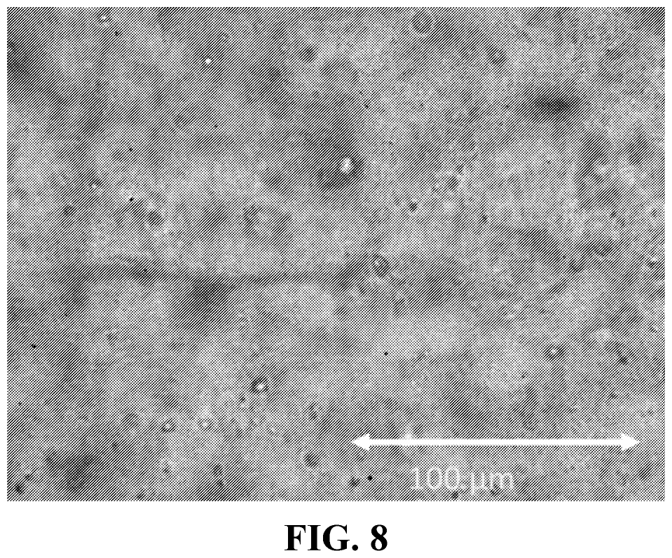

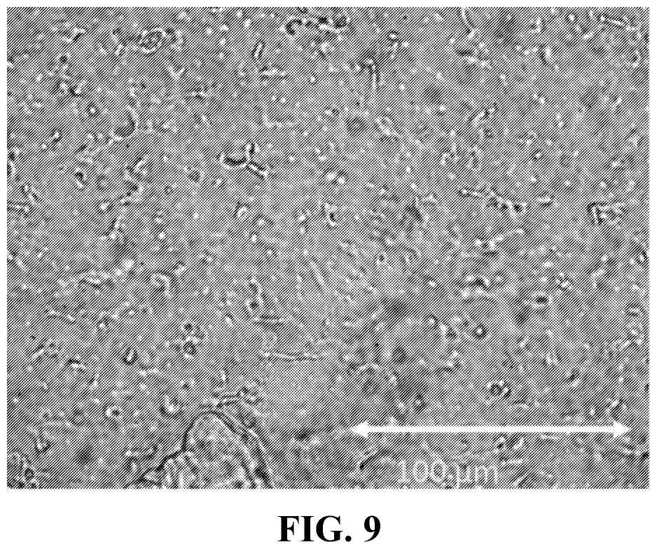

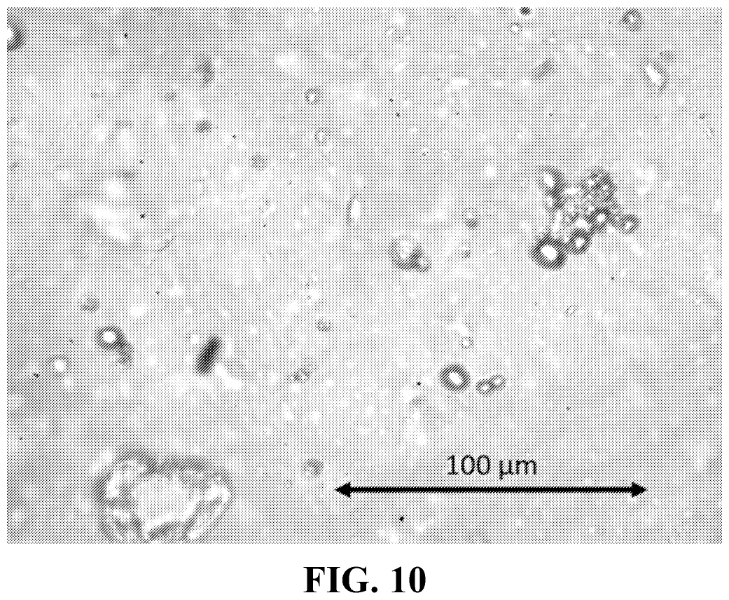

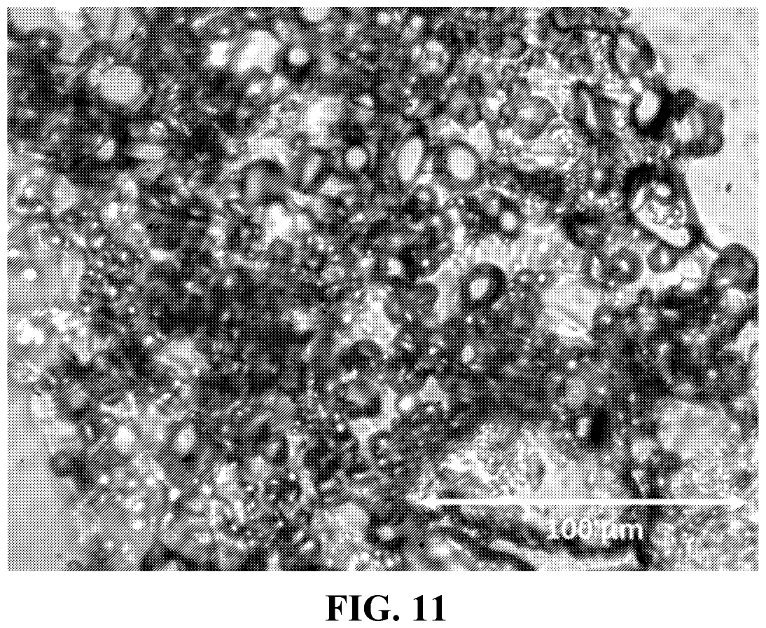

Urothelial cell scratch assays, as shown in FIG. 5, were designed to mimic the first 48 hours of urothelial ingrowth after injury or surgery. As used herein a “scratch assay,” (i.e., a wound assay) is a method to measure two-dimensional cell migration. Six growth factors were selected and tested: EGF, FGF-2, PDGF, TGF-β1, and VEGF. As shown in FIGS. 7A-7B, IGF-1 and EGF showed increase in wound gap coverage at 48 hours.

For reasons of completeness, various aspects of the technology are set out in the following numbered embodiments:

Embodiment 1. A stent, the stent comprising:
a thermoplastic polyurethane (TPU) material, and
a plurality of silk particles covalently attached to the TPU material, wherein the particles comprise a urological therapeutic agent.

Embodiment 2. The stent of embodiment 1, wherein the stent is a catheter.

Embodiment 3. The stent of embodiment 1 or 2, wherein the stent has an elastic modulus of 1 MPa to 20 MPa.

Embodiment 4. The stent of any one of embodiments 1-3, wherein the TPU material comprises 18% by weight (wt %) to 48 wt % poly (tetrahydrofuran).

Embodiment 5. The stent of any one of embodiments 1-4, wherein the silk particles are attached to the TPU material via an amide bond.

Embodiment 6. The stent of any one of embodiments 1-5, wherein the silk particles have an average diameter of 0.1 μm to 30 μm.

Embodiment 7. The stent of any one of embodiments 1-6, wherein the loading of the urological therapeutic agent is 0.001 mg to 0.4 mg of urological therapeutic agent per 1 mg of silk particles.

Embodiment 8. The stent of any one of embodiments 1-7, wherein the urological therapeutic agent is covalently conjugated to the silk particles.

Embodiment 9. The stent of any one of embodiments 1-8, wherein the urological therapeutic agent is a cell, a protein, a peptide, a nucleic acid, a nucleic acid analog, a nucleotide, an oligonucleotide, a peptide nucleic acid (PNA), an aptamer, an antibody, an antigen, an epitope, a hormone, a hormone antagonist, a growth factor, a recombinant growth factor, a cell attachment mediator, a cytokine, an enzyme, a small molecule, an antibiotic, an antimicrobial compound, a virus, an antiviral, or a combination thereof.

Embodiment 10. The stent of any one of embodiments 1-9, wherein the urological therapeutic agent comprises an alkylating agent, a platinum agent, an antimetabolite, a topoisomerase inhibitor, an antitumor antibiotic, an antimitotic agent, an aromatase inhibitor, a thymidylate synthase inhibitor, a DNA antagonist, a farnesyltransferase inhibitor, a pump inhibitor, a histone acetyltransferase inhibitor, a metalloproteinase inhibitor, a ribonucleoside reductase inhibitor, a TNF alpha agonist, a TNF alpha antagonist, an endothelin receptor antagonist, a retinoic acid receptor agonist, an immuno-modulator, a hormonal agent, an antihormonal agent, a photodynamic agent, or a tyrosine kinase inhibitor.

Embodiment 11. The stent of any one of embodiments 1-9, wherein the urological therapeutic agent comprises an aminoglycoside, bacitracin, a corbapenem, a cephalosporin, colistin, methenamine, a monobactam, a penicillin, polymyxin B, a quinolone, vancomycin, chloramphenicol, clindanyan, a macrolide, lincomyan, nitrofurantoin, sulfonamides, a tetracycline, trimethoprim, metronidazole, a fluoroquinolone, or ritampin.

Embodiment 12. The stent of any one of embodiments 1-9, wherein the urological therapeutic agent comprises edrophonium chloride, N-methylphysostigmine, neostigmine bromide, physostigmine sulfate, tacrine, tacrine, 1-hydroxy maleate, iodotubercidin, p-bromotetramiisole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N′-monomethyl-L-arginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4, 5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, or allopurinol.

Embodiment 13. The stent of any one of embodiments 1-9, wherein the urological therapeutic agent comprises pyrilamine, chlorpheniramine, or tetrahydrazoline.

Embodiment 14. The stent of any one of embodiments 1-9, wherein the urological therapeutic agent comprises a corticosteroid, a nonsteroidal anti-inflammatory drug, acetaminophen, phenacetin, a gold salt, chloroquine, D-penicillamine, methotrexate colchicine, allopurinol, probenecid, or sulfinpyrazone.

Embodiment 15. The stent of any one of embodiments 1-9, wherein the urological therapeutic agent comprises mephenesin, methocarbomal, cyclobenzaprine hydrochloride, trihexylphenidyl hydrochloride, levodopa/carbidopa, or biperiden.

Embodiment 16. The stent of any one of embodiments 1-9, wherein the urological therapeutic agent comprises atropine, scopolamine, oxyphenonium, or papaverine.

Embodiment 17. The stent of any one of embodiments 1-9, wherein the urological therapeutic agent comprises aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids, procaine, lidocaine, tetracaine, bupivacaine, ropivacaine, or dibucaine.

Embodiment 18. The stent of any one of embodiments 1-9, wherein the urological therapeutic agent comprises sodium fluorescein, rose bengal, methacholine, adrenaline, cocaine, atropine, alpha-chymotrypsin, hyaluronidase, betaxalol, pilocarpine, timolol, timolol salts, or combinations thereof.

Embodiment 19. The stent of any one of embodiments 1-9, wherein the urological therapeutic agent comprises imipramine, amitriptyline, nortriptyline, protriptyline, desipramine, amoxapine, doxepin, maprotiline, tranylcypromine, phenelzine, or isocarboxazide.

Embodiment 20. The stent of any one of embodiments 1-9, wherein the urological therapeutic agent comprises a platelet-derived growth factor (PDGP), a neutrophil-activating protein, a monocyte chemoattractant protein, a macrophage-inflammatory protein, a platelet factor, a platelet basic protein, a melanoma growth stimulating activity, an epidermal growth factor, a transforming growth factor alpha, fibroblast growth factor, platelet-derived endothelial cell growth factor, insulin-like growth factor, glial derived growth neurotrophic factor, ciliary neurotrophic factor, nerve growth factor, bone growth/cartilage-inducing factor alpha, growth/cartilage-inducing factor beta, a bone morphogenetic protein, an interleukin, an interferon, a hematopoietic factor, a tumor necrosis factor, or transforming growth factor beta.

Embodiment 21. The stent of any one of embodiments 1-9, wherein the urological therapeutic agent comprises an estrogen, an anti-estrogen, a progestin, an antiprogestin, an androgen, an anti-androgen, a thyroid hormone, or a pituitary hormone.

Embodiment 22. The stent of any one of embodiments 1-21, wherein the stent provides extended release of the urological therapeutic agent for up to 168 hours.

Embodiment 23. A catheter, the catheter comprising:
a thermoplastic polyurethane (TPU) material; and
a plurality of silk particles attached to the TPU material, wherein each particle comprises a urological therapeutic agent; and
wherein the catheter has an elastic modulus of 1 MPa to 20 MPa.

Embodiment 24. The catheter of embodiment 23, wherein the loading of the urological therapeutic agent is 0.001 mg to 0.4 mg of urological therapeutic agent per 1 mg of silk particles.

Embodiment 25. The catheter of embodiment 23 or 24, wherein the catheter provides extended release of the urological therapeutic agent for up to 168 hours.

Embodiment 26. A method of manufacturing a stent, the method comprising:
enriching a thermoplastic polyurethane (TPU) material with a plurality of carboxylic acid groups; and
covalently coupling a plurality of silk particles to the carboxylic acid groups, wherein the silk particles comprise a urological therapeutic agent.

Embodiment 27. The method of embodiment 26, wherein enriching the TPU material with a plurality of carboxylic acid groups comprises photooxidation of the TPU material with an oxidizing agent.

Embodiment 28. The method of embodiment 26 or 27, wherein covalently coupling the silk particles to the carboxylic acid groups comprises:
activating the carboxylic acid groups by reacting the groups with a carbodiimide; then
reacting the silk particles with the activated carboxylic acid groups.

Embodiment 29. The method of any one of embodiments 26-28, wherein the silk particles comprise 0.01 wt % to 99.99 wt % amine-functionalized silk.

Embodiment 30. The method of any one of embodiments 26-29, wherein the silk particles comprise 0.01 wt % to 99.99 wt % unfunctionalized silk.

Embodiment 31. A method for treating a urological disease or disorder, the method comprising:
implanting or inserting the stent of any one of embodiments 1-22 or the catheter of any one of embodiments 23-25 into a subject in need thereof.

Embodiment 32. A stent of any one of embodiments 1-22 or a catheter of any one of embodiments 23-25, for use in the treatment of a urological disease or disorder.

1. A stent, the stent comprising:
a thermoplastic polyurethane (TPU) material, and
a plurality of silk particles covalently attached to the TPU material, wherein the particles comprise a urological therapeutic agent.

2. The stent of claim 1, wherein the stent is a catheter.

3. The stent of claim 1, wherein the stent has an elastic modulus of 1 MPa to 20 MPa.

4. The stent of claim 1, wherein the TPU material comprises 18% by weight (wt %) to 48 wt % poly (tetrahydrofuran).

5. The stent of claim 1, wherein the silk particles are attached to the TPU material via an amide bond.

6. The stent of claim 1, wherein the silk particles have an average diameter of 0.1 μm to 30 μm.

7. The stent of claim 1, wherein the loading of the urological therapeutic agent is 0.001 mg to 0.4 mg of urological therapeutic agent per 1 mg of silk particles.

8. The stent of claim 1, wherein the urological therapeutic agent is covalently conjugated to the silk particles.

9. The stent of claim 1, wherein the urological therapeutic agent is a cell, a protein, a peptide, a nucleic acid, a nucleic acid analog, a nucleotide, an oligonucleotide, a peptide nucleic acid (PNA), an aptamer, an antibody, an antigen, an epitope, a hormone, a hormone antagonist, a growth factor, a recombinant growth factor, a cell attachment mediator, a cytokine, an enzyme, a small molecule, an antibiotic, an antimicrobial compound, a virus, an antiviral, or a combination thereof.

10. The stent of claim 1, wherein the urological therapeutic agent comprises an alkylating agent, a platinum agent, an antimetabolite, a topoisomerase inhibitor, an antitumor antibiotic, an antimitotic agent, an aromatase inhibitor, a thymidylate synthase inhibitor, a DNA antagonist, a farnesyltransferase inhibitor, a pump inhibitor, a histone acetyltransferase inhibitor, a metalloproteinase inhibitor, a ribonucleoside reductase inhibitor, a TNF alpha agonist, a TNF alpha antagonist, an endothelin receptor antagonist, a retinoic acid receptor agonist, an immuno-modulator, a hormonal agent, an antihormonal agent, a photodynamic agent, or a tyrosine kinase inhibitor.

11-21. (canceled)

22. The stent of claim 1, wherein the stent provides extended release of the urological therapeutic agent for up to 168 hours.

23. A catheter, the catheter comprising:
a thermoplastic polyurethane (TPU) material; and
a plurality of silk particles attached to the TPU material, wherein each particle comprises a urological therapeutic agent; and
wherein the catheter has an elastic modulus of 1 MPa to 20 MPa.

24. The catheter of claim 23, wherein the loading of the urological therapeutic agent is 0.001 mg to 0.4 mg of urological therapeutic agent per 1 mg of silk particles.

25. The catheter of claim 23, wherein the catheter provides extended release of the urological therapeutic agent for up to 168 hours.

26. A method of manufacturing a stent, the method comprising:
enriching a thermoplastic polyurethane (TPU) material with a plurality of carboxylic acid groups; and
covalently coupling a plurality of silk particles to the carboxylic acid groups, wherein the silk particles comprise a urological therapeutic agent.

27. The method of claim 26, wherein enriching the TPU material with a plurality of carboxylic acid groups comprises photooxidation of the TPU material with an oxidizing agent.

28. The method of claim 26, wherein covalently coupling the silk particles to the carboxylic acid groups comprises:
activating the carboxylic acid groups by reacting the groups with a carbodiimide; then reacting the silk particles with the activated carboxylic acid groups.

29. The method of claim 26, wherein the silk particles comprise 0.01 wt % to 99.99 wt % amine-functionalized silk.

30. The method of claim 26, wherein the silk particles comprise 0.01 wt % to 99.99 wt % unfunctionalized silk.

31. A method for treating a urological disease or disorder, the method comprising:
implanting or inserting the stent of claim 1 or the catheter of claim 23 into a subject in need thereof.

32. (canceled)
